SEQUENCE LISTING (8 sequences)

SEQ ID NO: 1 - DNA, 19 bp, Artificial Sequence
  Synthetic primer GAPDH-F
acatcgctcagacaccatg

SEQ ID NO: 2 - DNA, 22 bp, Artificial Sequence
  Synthetic primer GAPDH-R
tgtagttgaggtcaatgaaggg

SEQ ID NO: 3 - DNA, 20 bp, Artificial Sequence
  Synthetic primer PPARgamma-F
gagcccaagtttgagtttgc

SEQ ID NO: 4 - DNA, 22 bp, Artificial Sequence
  Synthetic primer PPARgamma-R
gcaggttgtcttgaatgtcttc

SEQ ID NO: 5 - DNA, 22 bp, Artificial Sequence
  Synthetic primer FADS2-F
tgtctacagaaaacccaagtgg

SEQ ID NO: 6 - DNA, 21 bp, Artificial Sequence
  Synthetic primer FADS2-R
tgtggaagatgttaggcttgg

SEQ ID NO: 7 - DNA, 20 bp, Artificial Sequence
  Synthetic primer TGFbetaRII-F
ctgtggatgacctggctaac

SEQ ID NO: 8 - DNA, 20 bp, Artificial Sequence
  Synthetic primer TGFbetaRII-R
catttcccagagcaccagag

(19) United States
(12) Patent Application Publication
(10) Pub. No.: US 2015/0268254 A1
(43) Pub. Date: Sep. 24, 2015

(54) SEBOCYTE CELL CULTURING AND METHODS OF USE

(71) Applicant: Children's Hospital Medical Center, Cincinnati, OH (US)

(72) Inventors: Geraldine Guasch, Cincinnati, OH (US); Adrian J. McNairn, Cincinnati, OH (US)

(21) Appl. No.: 14/433,507

(22) PCT Filed: Oct. 4, 2013
(86) PCT No.: PCT/US13/63367

§ 371 Date: Apr. 3, 2015

Related U.S. Application Data
(60) Provisional application No. 61/765,861, filed on Feb. 18, 2013.
(60) Provisional application No. 61/710,359, filed on Oct. 5, 2012.

(51) Int. Cl. G01N 33/92; C12Q 1/68; G01N 33/50; C12N 5/071

(52) U.S. Cl. CPC G01N 33/92; C12N 5/0633; C12Q 1/6883; G01N 33/5023; C12N 2533/12; C12N 2533/52; C12N 2500/84; C12Q 2600/136; C12Q 2600/158; C12N 2501/11; C12N 2501/33; C12N 2500/32; C12N 2501/30

(57) ABSTRACT

Methods of culturing sebocyte cells, isolated populations of sebocytes, and methods of using the cultured sebocyte cells for screening compounds that inhibit or activate lipogenesis are provided.

### TECHNICAL FIELD

The disclosure relates to methods of culturing sebocyte cells, and methods of using the cultured sebocyte cells for screening compounds that inhibit or activate lipogenesis.

### BACKGROUND OF THE DISCLOSURE

In humans, sebaceous glands are distributed throughout all the skin and found in greatest abundance on the face and scalp, and are only absent from the palms and soles. Sebaceous glands are microscopic glands which secrete an oily substance (sebum) in the hair follicles to lubricate the skin and hair of animals [1]. Their function with the epidermis is to prevent the skin from dehydration and protect the body against infections and physical, chemical and thermal assaults of the environment. The main components of human sebum are triglycerides and fatty acids (57.5%), wax esters (26%) and squalene (12%) [2]. The production of sebum is regulated throughout life, and decreases dramatically with age [3]. This is associated with increased dryness and fragility of the skin. Moreover, several human diseases, such as acne vulgaris, atopic dermatitis, seborrheic dermatitis and primary cicatricial alopecia are thought to be associated with deregulation of the sebaceous glands [2, 4, 5].

There is a crucial interdependency of sebaceous glands with hair follicles and epidermis as sebocyte dysfunction results in degeneration of hair follicle structures [5, 6] and a defective skin barrier. This is illustrated in the asebia mutant mouse, which lacks an enzyme that desaturates fatty acids. This mutant displays rudimentary sebaceous glands and alteration in the profile of skin surface lipids leading to chronic inflammatory reactions, alopecia and dermal scarring [6].

Successful growth of primary human cells often constitutes a breakthrough in a specific area of human biology with important clinical implications. Tissue stem cells such as those of the blood and the skin epidermis have already been successfully used in clinics for decades [7, 8]. In particular, epidermal cells (keratinocytes) can be cultured in vitro and can be efficiently manipulated to form a three dimensional epidermis [9, 10]. This model not only serves as a tool to treat patients with severe skin injury, but also provides the basic means to study the molecular mechanisms regulating human epidermal regeneration and differentiation.

Despite these advancements with other types of cells, successful methods for culturing human primary sebocytes are not available. Human sebaceous gland cell lines have been established in the past from adult human facial skin and periauricular area [11-14], but their immortalization with Simian virus-40 large T antigen or HPV16/E6E7 genes, which bypass the p53 and retinoblastoma protein mediated restriction point, results in cellular transformation that have limited their use for analyzing their cell cycle and differentiation regulation. Consequently, there remains a need for methods for culturing human primary sebocytes.

### SUMMARY OF THE DISCLOSURE

The present disclosure provides methods of culturing primary sebocyte cells comprising culturing sebaceous glands sandwiched between pieces of glass in cell culture medium suitable for culturing sebocytes for a length of time sufficient for formation of sebocyte cells on said sebaceous glands. The methods can further comprise removing the sebocyte cells from the sebaceous gland, and culturing the sebocyte cells on glass coated with an extracellular matrix protein, in a medium suitable for culturing sebocytes.

Further methods of the present disclosure for culturing primary sebocyte cells comprise culturing primary sebocyte cells on fibronectin coated glass in a medium comprising a basal medium, epidermal growth factor, cholera toxin, adenine, insulin, hydrocortisone, fetal bovine serum, and antibiotic/antimitotic.

Another aspect of the present disclosure provides an isolated population of cultured sebocyte cells obtained by a method of the present disclosure.

An additional aspect of the present disclosure provides methods for identifying compounds that regulate lipogenesis, or determining the effect of a test compound on lipogenesis, comprising a) adding a test compound to a population of cultured sebocyte cells obtained by a method of the present disclosure, and b) measuring the effect of the test compound on lipid production in the sebocyte cells.

These and other aspects of the present disclosure are set out in the appended claims, and described in greater detail in the detailed description of this disclosure.

### BRIEF DESCRIPTION OF THE DRAWINGS

FIGS. 1a-1c show photographs of sebaceous glands and 1d shows the method of isolation

FIGS. 2a-h show graphs of gene expression involved in lipid analysis.

FIGS. 3a-3c show photographs of sebaceous glands.

FIGS. 4a-4f show graphs of the effects of TGFβ signaling on sebocyte differentiation.

FIGS. 5a-5d show photographs of the effects of inhibition of TGFβ signaling in sebocytes stably expressing a shRNA against TGFβRII.

FIG. 6 shows a diagram of a human sebaceous gland and hair follicle.

FIGS. 7a-7g show photographs of expression of markers of sebaceous gland differentiation in human scalp, breast, chest, and facial tissues.

FIGS. 8a-8c show photographs of primary sebocytes and lipid content before and after treatment with linoleic acid.

FIGS. 9a and 9b show graphs of fatty acid desaturase 2 (FADS2) and PPARγ expression in sebocytes derived from breast or facial skin.

FIGS. 10a and 10b show inhibition of TGFβ signaling in primary SSG3 cells.

### DETAILED DESCRIPTION OF THE DISCLOSURE

The present disclosure provides methods of cultivating human primary sebocytes, without transformation, and using a feeder layer-free culture system. The novel methods of culturing and successfully passaging human primary sebocytes overcome a major hurdle in the field of epithelial cell culture.

In the practice of the methods of the present disclosure, sebaceous glands are excised from a sample of skin from a donor, and cultured for a sufficient time until sebocytes form on the gland.

Sebaceous glands can be excised from the skin sample by any suitable process. For example, the skin sample can be cut into small pieces and treated with dispase to separate epidermis from dermis. After dispase treatment, intact sebaceous glands are isolated with microsurgical instruments under a dissecting microscope. Preferably, the hair shaft and a small amount of tissue are retained with the sebaceous gland to preserve the microenvironment around the gland.

The skin sample used in the methods of the present disclosure can be obtained from any location on the human body where the skin contains sebaceous glands. Primary sebocyte cultures derived scalp, breast, chest, and face skin, have been established. Preferably, the sebocytes are from human pediatric donors (donors ranging in age from newborn to less than about fifteen years old).

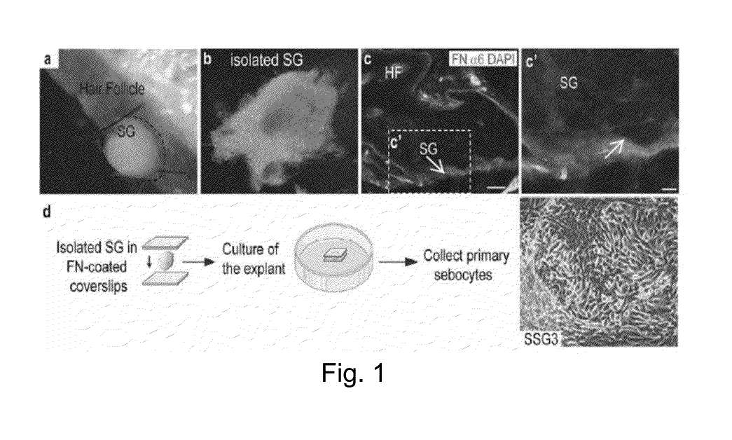

The explant containing the sebaceous gland is then sandwiched between pieces of glass, such as glass coverslips, as shown in FIG. 1d. Preferably, the glass is coated with an extracellular matrix protein. The preferred extracellular matrix protein is fibronectin, preferably human fibronectin.

Glass coverslips covered with fibronectin, also referred to herein as fibronectin coated glass and similar terms, are prepared using conventional methods. The amount of human fibronectin in the coating can vary, but is preferably about 10 μg/ml. The coverslips can be coated with fibronectin by adding the fibronectin on the coverslips, and leaving the coverslips for one hour at room temperature or overnight at 4° C. The coverslips are then washed three times with PBS 1× buffer and stored at 4° C. for no longer than one week.

The sebaceous gland explants sandwiched between glass coverslips are then cultured in sebocyte medium until sebocytes grow out of the sebaceous glands. Sebocytes become apparent from the periphery of the sebaceous gland lobules after about one to two weeks of culturing the explants. The cells are cultured in a 37° C. incubator with 5% carbon dioxide.

Sebocyte cell culture medium is a cell culture medium suitable for culturing sebocytes, including sebocyte cell culture media known in the art.

The preferred sebocyte medium comprises a basal medium, DMEM/Ham's F-12 (3:1), supplemented with Epidermal Growth Factor (EGF 3 ng/ml, Austral Biologicals San Ramon, Calif.), cholera toxin (1.2×10⁻¹⁰M, Sigma Chemical Co, St. Louis, Mo.), adenine (24 μg/ml, Sigma), insulin (10 ng/ml Sigma), hydrocortisone (45.2 ng/ml, Sigma), fetal bovine serum (FBS) (2.5% Hyclone, Logan, Utah), antibiotic/antimitotic (Penicillin/streptomycin) (100×, Invitrogen, Carlsbad, Calif.), as described in [12].

The combination of the antibiotics penicillin and streptomycin are used to prevent bacterial contamination of cell cultures due to their effective action against gram-positive and gram-negative bacteria, respectively. Amphotericin B is used to prevent fungal contamination of cell cultures due to its inhibition of multicellular fungus and yeast.

After sebocytes form on the sebaceous gland explants, the sebocytes are removed from the explants, and the isolated cells cultured on fibronectin-coated glass, such as a glass coverslip. To expand the cells, trypsin-EDTA (GIBCO, Carlsbad, Calif.) 0.05% in phosphate-buffered saline solution (PBS) was used to detach the cells. The sebocyte cells were put in culture in a new plate coated with fibronectin 10 μg/ml in a 12 mm plate. The cells can be passaged up to about ten times, after which the cells will undergo senescence. Preferably low passage P2-P6 cells are used in the methods disclosed herein. When the sebocytes are expanded they do not need to be placed between 2 coverslips. When cells reach 80-90% confluence they can be expanded. A density of 20-30% is preferable for culturing the cells. Sebocyte cells can grow on plastic without a fibronectin-coated coverslip after few passages, but culturing the cells on fibronectin-coated coverslips is preferable.

Thus, the present disclosure provides methods for method of culturing primary sebocyte cells comprising culturing sebaceous glands sandwiched between pieces of glass in cell culture medium suitable for culturing sebocytes for a length of time sufficient for formation of sebocyte cells on said sebaceous glands. The methods can further comprise removing the sebocyte cells from the sebaceous gland, and culturing the sebocyte cells on glass coated with an extracellular matrix protein, in a medium suitable for culturing sebocytes.

The methods can also include the steps of obtaining a sample of skin, and removing sebaceous glands from the skin sample, which steps are performed prior to prior culturing the sebaceous glands.

The disclosure also provides methods of culturing primary sebocyte cells comprising culturing primary sebocyte cells on fibronectin coated glass in a medium comprising a basal medium, epidermal growth factor, cholera toxin, adenine, insulin, hydrocortisone, fetal bovine serum, and antibiotic/antimitotic.

Another aspect of the present disclosure provides isolated populations of sebocytes obtained by the methods of the present disclosure. As used herein, an isolated population of sebocytes refers to sebocytes removed from their native location in a sebaceous gland. The population of sebocyte cells is cultured, and preferably contains only undifferentiated and/or differentiated sebocytes.

The sebocytes can be characterized using the markers disclosed in the Examples and discussed below, as well as other markers known in the art. One of the primary cultures derived from scalp (called SSG3) has been extensively characterized. The cells express markers of sebaceous gland differentiation, such as PPARγ [26] and [23], Blimp1 [26], c-Myc, Keratin 7 and the epithelial membrane antigen EMA/Muc1. The cultured sebocytes can differentiate in vitro after linoleic acid treatment. Cytosolic lipid droplets were detected in untreated cells and an increase of lipid droplets with higher electron density after linoleic acid treatment was readily detected by electron microscopy. Fatty acid desaturase 2 (FADS2), a unique Δ6 desaturase involved in the linoleic acid metabolism and sebum production [29], is highly expressed at the mRNA level in the transformed sebocytes SEB-1 and in SSG3 cells compared to human keratinocytes NIKS. FADS2 is detectable mainly in differentiated sebocytes that have reached lipid synthetic capacity, providing a functional marker of activity and differentiation in sebocytes [49]. Differentiation of the cultured sebocytes induced by linoleic acid treatment is followed by an increase in PPARγ and FADS2 expression, in contrast to human keratinocytes and SEB-1 that do not show any significant changes. Those results were further confirmed by the analysis of the neutral and polar lipids in the primary SSG3 cells. As expected, the composition of phospholipids, the major components of cell membranes, is similar between the NIKS and SSG3 cells. However, there are differences in the composition of fatty acids. The composition of two specific acids, sapienic acid and palmitoleic acid, which are synthesized by two different desaturases, Δ6/FADS2 and Δ9 respectively [31], was analyzed. Sapienic acid, dominantly found in sebum in vivo, is detected only in SSG3 cells (2.15%) compared to NIKS (0.79%). In contrast, the major fatty acid found in NIKS is palmitoleic acid (6.95%) compared to SSG3 cells (1.2%). Moreover, the ratio Δ6/Δ9 desaturase (% Sapienic acid/% Palmitoleic acid), an index of sebocyte maturation [32], is largely superior in SSG3 cells compared to the NIKS keratinocytes (178.9 and 11.42 respectively), reflecting the functionality of the scalp-derived sebocytes. The populations of sebocytes produced by the methods of the present disclosure not only express genes involved in sebum production and lipid synthesis, but they can also produce sebum-characteristic lipids.

As shown herein, Transforming Growth Factor β (TGFβ) signaling plays a key role in maintaining the sebocytes in an undifferentiated state. Activation of the TGFβ signaling pathway downregulates the expression of genes involved in the production of characteristic sebaceous lipids (PPARγ and FADS2) but does not affect the proliferation of human sebocytes. Moreover, repression of TGFβ signaling through knockdown of the TGFβ Receptor II (TGFβRII) causes increased lipid production in those cells detected by Nile red and Oil red O staining. This increase in lipid production after blocking TGFβ signaling has been confirmed by electron microscopy. These observations highlight the relevance of this previously unidentified pathway in human sebaceous gland biology.

Primary SG cells do not express keratin 8 in contrast to previously immortalized sebocytes. Keratin 8 is not normally expressed in normal sebaceous gland in vivo [25] and the results herein indicate that the transformation process in the immortalized line has likely altered the expression of several fundamental cell markers. Moreover, primary sebocytes and the immortalized cells showed different responsiveness to linoleic and TGFβ1 treatment suggesting that the cell properties of those cells substantially differ.

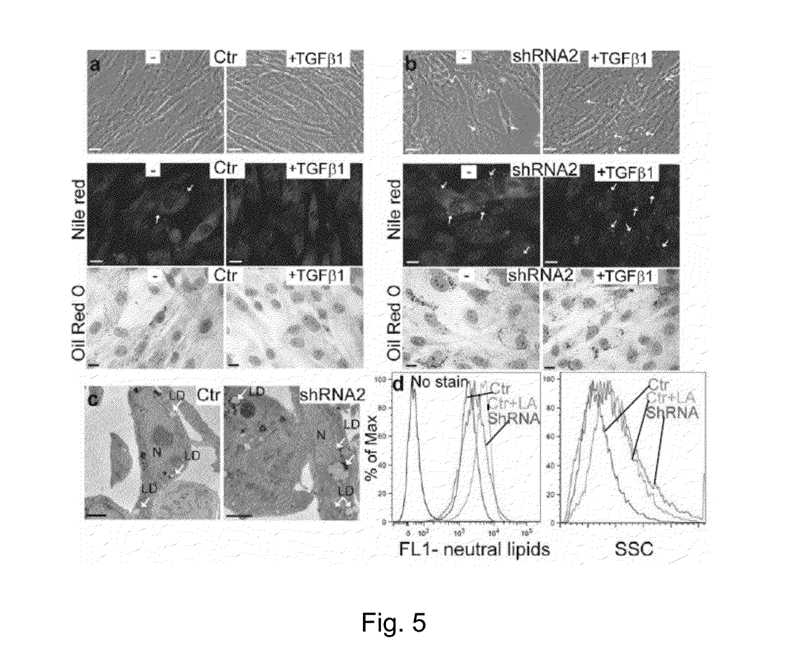

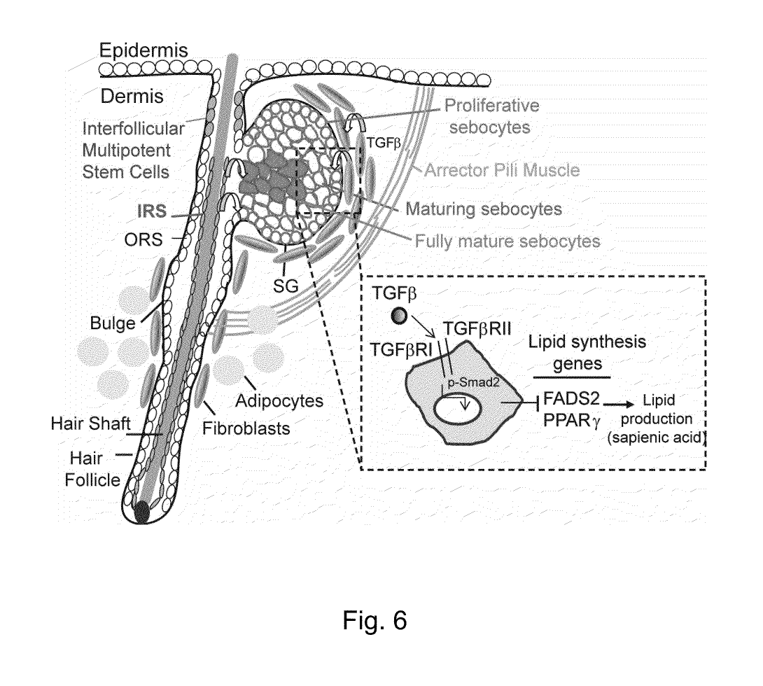

While not wishing to be bound by any specific theory or mode of action, it is presently believed that TGFβ signaling maintains sebocytes in an undifferentiated state by decreasing the expression of FADS2 and PPARγ, thereby decreasing lipid accumulation through the TGFβRII-Smad2 dependent pathway (FIG. 5). Molecular crosstalk between the dermis and the epithelial cells are crucial for the initiation and maintenance of the hair follicles [37]. It seems most likely that similar mechanisms of communication between sebocytes and the surrounding dermal tissue exist. In mouse, inner root sheath of the hair follicle released TGFβ1 and a bidirectional interaction between sebocytes and hair follicle epithelium could be envisioned [17]. In the dermis, human fibroblasts secrete TGFβ [38, 39] which may then act on keratinocytes and sebocytes (FIG. 6). Another component in the microenvironment that could also be part of this crosstalk are the arrector pili muscle cells recently shown to be controlled by bulge stem cells in mouse [40]. Being located in close proximity to the sebaceous gland, arrector pili muscles could help release sebum onto the skin surface [41].

Impairment of the skin barrier due to the deregulation of sebum production when associated with bacteria colonization and inflammation, can be the cause of serious skin condition in human. For instance, hyperseborrhea combined with the presence of Propionibacterium acnes and inflammation can lead to acne vulgaris [2] and Staphylococcus aureus can aggravate atopic dermatitis [4]. Sebocytes can produce antimicrobial peptides such as defensin-1 and -2 upon exposure to Propionibacterium acnes or lipopolysaccharides [42, 43] to prevent from bacteria colonization [44] and from an upregulation of sebum production [45]. Studies have revealed that TGFβ induces the expression of human β-defensin-2 in endothelial cells [46] and influences inflammatory response [47]. With the novel methods for culturing sebocytes described herein, different interactions with the microenvironment can now be investigated.

A further aspect of the present disclosure provides methods for identifying compounds that regulate lipogenesis, or determining the effect of a test compound on lipogenesis. The methods comprise a) adding a test compound to a population of cultured sebocyte cells obtained by the methods disclosed herein, and b) measuring the effect of the test compound on lipid production in the sebocyte cells. The primary sebocytes can be used to measure the effect of test compounds known to be inhibitors or activators of lipogenesis, and identify test compounds that inhibit or activate lipogenesis, or change alter the effects of an inhibitor or activator of lipogenesis. The test compound can be any type of chemical compound. Examples of known inhibitors or activators of lipogenesis include 5α-DHT (dihydrotestosterone), 5-DHEA (5-dehydroepiandrosterone), cyproteron acetate, estradiol, dexamethasone, isotretinoin, and rosiglitazone. Suitable test compounds include androgens, antiandrogens, estrogens, corticoids, retinoids, PPAR agonists, 5α-reductase inhibitors, and TGFβ1 ligand.

The effect on lipid production can be measured, for example, according to the method described in Example 3, or any other method known in the art for detecting lipid production.

A test compound is added to a population of sebocytes, obtained according to the methods of the present disclosure, and the effect of the compound on lipogenesis is determined. Suitable markers for determining lipid production include: analysis of expression of FADS2 and PPARγ after treatment of the cells with linoleic acid, quantification of neutral lipid accumulation (representative of sebum lipids) and the presence of polar lipids (representative of phospholipids), as described in Example 3. FACS analysis as shown in FIG. 5D can be used to quantify neutral lipids.

Compounds identified using the methods of the present disclosure can be used in the same manner as compounds known in the art to affect lipogenesis, for treatment of skin conditions.

The disclosure also provides methods for identifying compounds that regulate proliferation of sebocytes, differentiation of sebocytes, cell cycle of sebocytes, or survival of sebocytes. The methods comprise a) adding a test compound to a population of cultured sebocyte cells obtained by the methods disclosed herein, and b) measuring the effect of the test compound on proliferation of the sebocytes, differentiation of the sebocytes, cell cycle of the sebocytes, or survival of the sebocytes. The primary sebocytes can be used to measure the effect of test compounds known to regulate proliferation, differentiation, cell cycle or survival of sebocytes, and identify test compounds that regulate proliferation of sebocytes, differentiation of sebocytes, cell cycle of sebocytes, or survival of sebocytes. The test compound can be any type of chemical compound. Proliferation can be follow by bromodeoxyuridine (BrdU) and Ki67 labeling, immunofluorescence and Flow Cytometry assays. Cell cycle analysis can be performed using 7AAD and Brdu and analyzed by Flow Cytometry. Survival can be follow by cell counting.

The following non-limiting examples are presented to further facilitate an understanding of the present disclosure.

### EXAMPLES

### Example 1

### Method of Culturing Sebocyte Cells

Sebaceous gland populations were generated from human scalp (SSG3), face, and breast from both male and female donors ranging in age from 9 months to 12 years old. The skin samples were collected as a surgical waste with information provided regarding the age and sex of the donors with Institutional Review Board (IRB) approval at Cincinnati Children's Hospital Medical Center.

After cutting the skin samples in small pieces, the sample was treated with dispase 1× (2 mg/ml in PBS1×, Gibco/Invitrogen cat#17105-04; Carlsbad, Calif.) overnight at 4° C. at before dissection. The dispase is used to separate epidermis from dermis, and avoid epidermal cell contamination.

After treating the skin with dispase 1× (FIG. 1), intact sebaceous glands were isolated with microsurgical instruments under a dissecting microscope. The hair shaft and a small amount of tissue were retained with the sebaceous gland to preserve the microenvironment around the gland.

To mimic the microenvironment of the sebaceous gland, the explants were sandwiched between glass coverslips coated with human fibronectin (10 μg/ml, Millipore, Billerica, Mass.).

To coat the coverslips, the fibronectin was added on the coverslips at 10 μg/ml, 1 hour at room temperature or overnight at 4° C. The coverslips are then washed 3 times with PBS 1× and stored at 4° C. for no longer than one week.

The explants were cultivated in sebocyte medium as described (DMEM/Ham's F-12 (3:1), Epidermal Growth Factor (EGF 3 ng/ml, Austral Biologicals, San Ramon, Calif.), cholera toxin (1.2×10-10M, Sigma), adenine (24 μg/ml, Sigma), insulin (10 ng/ml Sigma), hydrocortisone (45.2 ng/ml, Sigma), FBS (2.5% Hyclone, Logan, Utah), antibiotic/antimitotic (100×, Invitrogen, Carlsbad, Calif.) in [12].

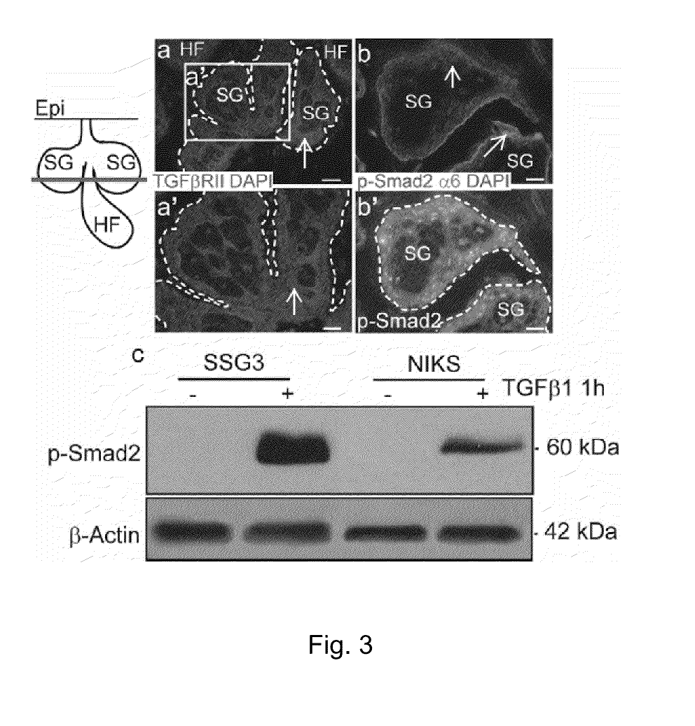

After 1-2 weeks of growth in culture, cellular outgrowth became apparent from the periphery of the gland lobules (FIG. 3). The explants were removed and the isolated cells cultured on the fibronectin-coated coverslips. Cells growing out of the periphery of the explants were isolated and cultured on fibronectin-coated coverslips. To expand the cells, trypsin-EDTA (GIBCO, Carlsbad, Calif.) 0.05% in phosphate-buffered saline solution (PBS) was used to detach the cells. The cells were then put in culture on a new plate coated with fibronectin 10 μg/ml in a 12 mm plate.

### Example 2

### Characterization of Cultured Sebocyte Cells

### Methods

### Western Blotting

Proteins were separated by electrophoresis on 10-12% acrylamide gels, transferred to nitrocellulose membranes and subjected to immunoblotting. Membranes were blocked for one hour with 5% non-fat milk or 5% BSA in PBS containing 0.1% Tween-20. Primary antibodies were generally used at a concentration of 1/1,000 and HRP-coupled secondary antibodies were used at 1/2,000 in 5% non-fat milk. Immunoblots were developed using standard ECL (Amersham, Pittsburgh, Pa.) and Luminata TM crescendo and classico (Millipore). Two-color immunoblot detection was performed using LI-COR Odyssey CLx (LI-COR Biosciences, Lincoln, Nebr.). Membranes were blocked in Odyssey blocking buffer (LI-COR) and secondary antibodies conjugated to IRDye 680LT and 800CW were used (1/10,000; LI-COR). Protein levels were quantified using the Odyssey Infrared Imaging System (LI-COR).

### Retroviral Infection

To ablate TGFβRII in SSG3 cells, shRNA vectors from the CCHMC Heart Institute lenti-shRNA library core (shRNA TGFβRII #197031 and 194992 and a shRNA control) were used. The human library was purchased from Sigma-Aldrich (MISSION shRNA; St. Louis, Mo.). Viral vector was produced by the Viral Vector Core at the Translational Core Laboratories, Cincinnati Children's Hospital Research Foundation. Cells were grown to 80% confluency in 6-well plates before being infected with the lentivirus for 48 h. Infected cells were selected with 1 pg/ml puromycin (Sigma) for 48 h. Following selection, TGFβRII knock down cells were grown in regular media for 48 h before being activated with 5 ng/ml TGFβ1 for 24 h.

### Histology and Immunofluorescence

Human tissues were frozen unfixed in OCT compound (Tissue-Tek, Sakura, Torrance, Calif.) for cryosectioning. Immunostainings were performed as previously described [48].

### Antibodies

Primary antibodies against the following proteins were used at the dilution indicated: PPARγ (Santa-Cruz Biotechnology Inc., Santa Cruz, Calif., H-100 1/250 for immunofluorescence, 1/500 for western blot), Blimp1 (Cell Signaling, Danvers, Mass., 1/500 for immunofluorescence, 1/1,000 for Western Blot), Fibronectin (Santa-Cruz Biotechnology Inc., Santa Cruz, Calif., EP5 1/150), Muc1 (Millipore, 1/500), cMyc (Cell Signaling, 1/800 for immunofluorescence, 1/1,000 for Western Blot), TGFβRII (Santa-Cruz Biotechnology Inc., Santa Cruz, Calif., sc-220 1/1,000), p-Smad2 (Cell Signaling, 1/100 for immunofluorescence, 1/1,000 for western blot), Smad2/3 (BD Biosciences, San Jose, Calif., 1/500), a6 integrin (CD49f, BD Biosciences, San Jose, Calif., 1/100), bromodeoxyuridine BrdU (Abcam, Cambridge, Mass., 1/500), Keratin 8 (this antibody, developed by Dr. Brulet and Dr. Kemler, was obtained from the NICHD Developmental Studies Hybridoma Bank maintained by the University of Iowa, 1/1,000), 8-actin (Sigma, 1/2,000), Keratin 7 (Cell Signaling, 1/1,000), 4′,6-diamidino-2-phenylindole (DAPI) was utilized as a marker of cell nuclei (Sigma Chemical Co., St. Louis, Mo., 1/5,000). Secondary antibodies Alexa Fluor 488 or 555 (Molecular Probes, Carlsbad, Calif.) were used at a dilution of 1/1,000. Fluorescence images were acquired with a fluorescent microscope Axiolmager M1 (Zeiss) and pictures were taken with an axioCam MRm camera (Zeiss, Thornwood, N.Y.).

### Real-Time PCR

Total RNA was isolated using a Rneasy Mini Kit (Qiagen, Germantown, Md.) and used to produce cDNA (Maxima first strand cDNA synthesis kit, Fermentas, Waltham, Mass.). Reverse transcription (RT) reactions were diluted to 10 ng/pl and 1 pl of each RT was used for real-time PCR. Real-time PCR was performed using the CFX96 real-time PCR System, CFX Manager Software and the SsoFast EvaGreen Supermix reagents (Biorad, Hercules, Calif.). All reactions were run in triplicate and analyzed using the AACT method with relative expression normalized to GAPDH.

| Primers used: | |
|---|---|
| (SEQ ID NO: 1) | |
| GAPDH-F: | ACATCGCTCAGACACCATG, |
| (SEQ ID NO: 2) | |
| GAPDH-R: | TGTAGTTGAGGTCAATGAAGGG |
| (SEQ ID NO: 3) | |
| PPARγ-F: | GAGCCCAAGTTTGAGTTTGC, |
| (SEQ ID NO: 4) | |
| PPARγ-R: | GCAGGTTGTCTTGAATGTCTTC, |
| (SEQ ID NO: 5) | |
| FADS2-F: | TGTCTACAGAAAACCCAAGTGG, |
| (SEQ ID NO: 6) | |
| FADS2-R: | TGTGGAAGATGTTAGGCTTGG, |
| (SEQ ID NO: 7) | |
| TGFβRII-F: | CTGTGGATGACCTGGCTAAC, |
| and | |
| (SEQ ID NO: 8) | |
| TGFβRII-R: | CATTTCCCAGAGCACCAGAG. |

### Lipogenesis Assays

For Nile red staining, cells or OCT sections were fixed 10 minutes at room temperature in 4% formaldehyde. After 3 wash in 1×PBS, staining with 0.1 pg/ml of Nile red (Sigma) was performed in 0.15M NaCl for 15 minutes at room temperature. For Oil red 0 staining, cells were fixed 15 minutes in 10% formalin, wash with water for 10 minutes and 60% isopropanol before being stained with Oil red 0 (0.7% in 60% isopropanol) for 45 minutes. Cells were rinsed with 60% isopropanol and the nuclei stained with haematoxylin. To trigger differentiation of sebocytes in vitro, linoleic acid (Sigma, 0.1 mM) was added directly to sebocyte media. To prepare cells for extraction of lipids, 20-30 millions of cells were pelleted, washed with 1×PBS and lipid were preserved in the dark at −80° C. under argon until analysis. The qualitative and quantitative composition of lipids in scalp-derived human sebocytes was determined using an Agilent 5973N Gas chromatograph/Mass spectrometer with a SPE cartridge (solid phase extraction) and was performed by Synelvia S.A.S (Labege, France).

### Nile Red Analysis by FACS

Cells were cultured in 6-well plates at 80% confluence and infected with the lentivirus expressing the shRNAs as previously described. After puromycin selection for 48 h, cells were washed in 1×PBS and treated with working medium with or without Linoleic acid (0.1 mM) for 24 h. The cells were trypsinized, washed once with 1×PBS and neutral lipids were labeled with the fluorescent dye Nile red (1 pg/ml in PBS). 10,000 cells per sample were analyzed using a FACS Canto I (BD Biosciences) equipped with a blue laser (488 nm excitation).

### Electron Microscopy

Cells were grown at 80% confluency in sebocyte media and rinsed once with 0.175M sodium cacodylate buffer. Cells were fixed in 3% glutaraldehyde/0.175M cacodylate buffer for 1 hour at 4° C. Dishes were washed twice with 0.175M sodium cacodylate buffer. Cells were post fixed in 1% osmium tetroxide/cacodylate buffer for 1 hour at 4° C. before being washed three times with 0.175M sodium cacodylate buffer. After the final wash with 1.5 ml, cells were scraped and centrifuged for 5 min at 10K. The cell pellet was then resuspended in 1 ml 1% agarose (Type IX ultra-low gelling tempt, Sigma) overnight at 4° C. The samples were then processed through a graded series of alcohols, infiltrated and embedded in LX-112 resin (Ladd Research, Williston, Vt. After polymerization at 60° C. for three days, ultrathin sections (100 nm) were cut using a Reichert-Jung Ultracut E microtome and counterstained in 2% aqueous uranyl acetate and Reynolds lead citrate. Images were taken with a transmission electron microscope (Hitachi H-6750) equipped with a digital camera (AMT 2k×2K tem CCD).

### Statistics

Data are expressed as means+/−SD. Comparison between two cell types was performed using unpaired two-tailed student's t test. Paired two-tailed student's t test was used when comparing the effect of a treatment on the same cell type. P<0.05 was considered significant.

### Results

### A.1 Primary Sebocytes Established from Pediatric Donors Express Markers of Sebaceous Gland Differentiation

To determine the pathways that regulate primary human sebocytes growth and differentiation, sebocytes were isolated and propagated by mimicking the microenvironment of the sebaceous glands in vitro Skin explants from donors ranging from 9 months to 12 years of age were microdissected, and the sebaceous glands were placed between fibronectin-coated glass coverslips to reproduce an in vivo environment. Using this technique, primary sebocyte cultures were derived from eight donors representing four skin tissue types: five scalp, one breast, one chest, and one face sample. All experiments were performed on passage 2 and later passages (3 to 5) without the use of extracellular matrix or supporting irradiated fibroblasts.

To verify that the cell cultures were indeed sebocytes, expression of known sebocyte markers was examined. Immunofluorescence staining and western blot demonstrated that those cells homogeneously express peroxisome proliferator-activated receptor gamma (PPARγ), an adipogenic transcription factor expressed in differentiating sebocytes [23], in vitro and in vivo, but not in human keratinocytes (NIKS) [24]. SSG3 cells express this sebocyte marker. Real-time PCR confirmed that primary SSG3 expressed a similar level of PPARγ as the immortalized sebocyte line SEB-1 [12]. However, in contrast to SEB-1, SSG3 cells do not express Keratin 8, not typically expressed in sebaceous gland in vivo [25]. Additionally, SSG3 cells express other markers of sebocytes such as Blimp1 and epithelial membrane antigen EMA/Muc1. In agreement with recent reports [13, 26], in human scalp sections from which SSG3 cells were derived, Blimp1 is expressed in terminally differentiated cells of the sebaceous glands and in the inner root sheath of the hair follicle. All the results shown in scalp-derived sebocytes have been confirmed to be similar in the breast and face derived-sebocytes. The only exception is the expression of Keratin 7, a marker of the undifferentiated sebocytes, detected at higher expression in protein lysates of the face-derived sebocytes compared to the scalp and the breast. The difference in Keratin 7 expression may depend on the location of where the cells derived. The established primary human sebocytes from Example 1 express typical sebocyte markers and represent a good model for studying sebocyte function.

### A.2 Primary Sebocytes can Differentiate In Vitro

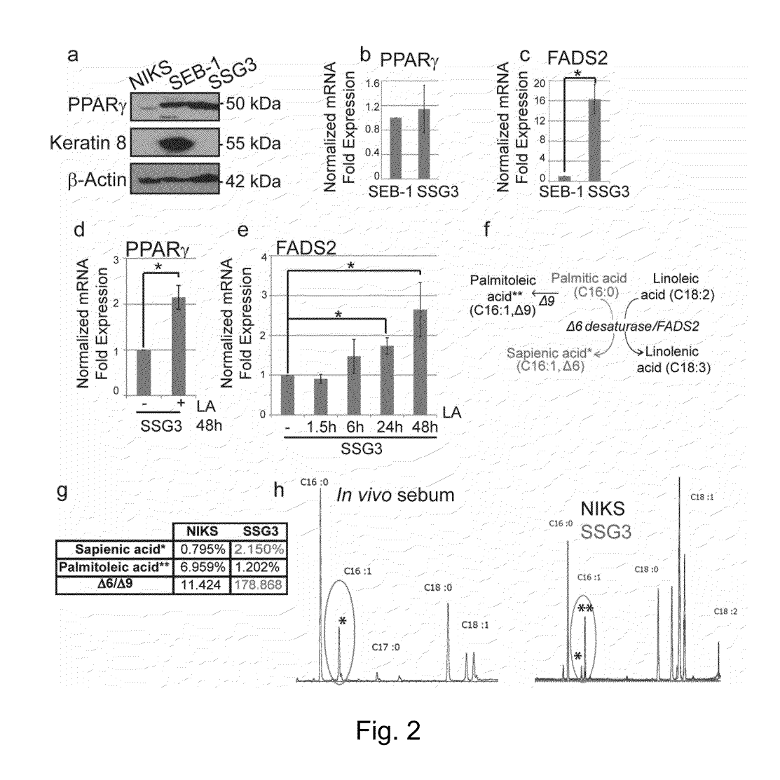

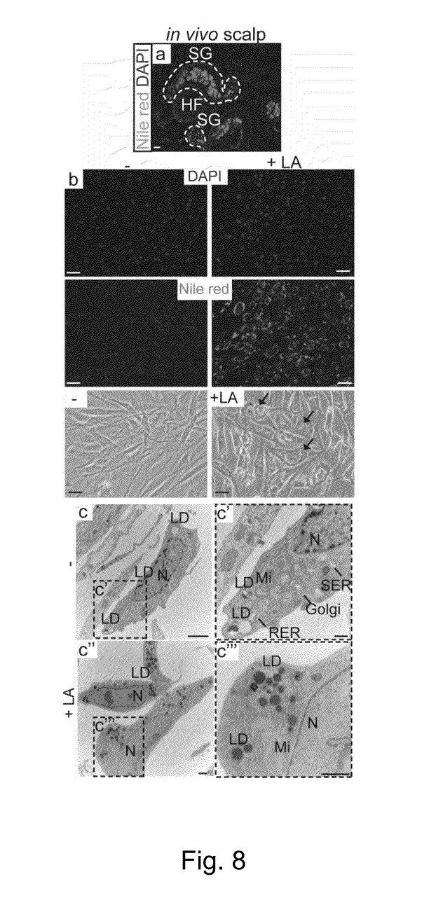

To confirm that the primary human sebocytes are functional in vitro, their ability to differentiate and produce human-specific lipids was analyzed. The lipophilic dye Nile red can be used to stain terminally differentiating sebocytes [27] (FIG. 8a). Linoleic acid is an essential polyunsaturated fatty acid that is used for biosynthesis of some prostaglandins and other polyunsaturated fatty acids and triggers the differentiation of sebocytes in vitro [28]. We therefore analyzed the cellular lipid distribution by Nile red stains after two days of linoleic acid treatment at physiological levels and show that SSG3 were indeed producing lipids (FIG. 8b). Moreover, cytosolic lipid droplets were detected by electron microscopy in untreated cells (FIG. 8c) as well as an increase of lipid droplets with higher electron density after linoleic acid treatment (FIG. 8c″). Humans possess a unique 46 desaturase/FADS2 gene [29] involved in the linoleic acid metabolism and sebum production. FADS2 is detectable mainly in differentiated sebocytes that have reached lipid synthetic capacity, providing a functional marker of activity and differentiation in sebocytes. It has been found according to this disclosure that FADS2 is highly expressed in SSG3 cells compared to SEB-1 (FIG. 2c). These results demonstrate that the SSG3 cells exhibit gene expression patterns characteristics of cells involved in sebocyte differentiation. Moreover, it has been shown that differentiation induced by linoleic acid treatment in SSG3 cells is followed by an increase in PPARγ, in contrast to SEB-1 that do not show any significant changes (FIG. 2d) and an increase in FADS2 in SSG3 (FIG. 2e).

Those data were further confirmed by the analysis of lipids in the primary SSG3 cells. Differences in the composition of fatty acids in particular sapienic acid, predominantly found in sebum in vivo [29], and palmitoleic acid have been found. They are synthetized by two desaturases, A6/FADS2 and A9 respectively [31] (FIG. 2f). The desaturation in A6 position instead of A9 is specific to human sebum [31]. Sapienic acid is detected only in SSG3 cells (2.150%) compared to NIKS (0.795%). In contrast, palmitoleic acid is predominantly found in NIKS (6.959%) compare to SSG3 cells (1.202%) (FIGS. 2g and h). Next, to determine the functionality of SSG3 cells, the ratio of 16/19 desaturase that is an index of sebocyte maturation and associated metabolic process [32] was quantified. It was found that this ratio in SSG3 cells is largely superior to the NIKS (178.868 and 11.424 respectively) reflecting the functionality of the scalp-derived sebocytes (FIG. 2g). The lipid analysis also revealed that only fatty acids with even-numbered carbon chains are present in SSG3 as in sebum in vivo (FIG. 2h). It can be concluded that the primary human sebocyte cultures that have been established according to this disclosure not only express genes involved in sebum production and lipids synthesis but can also produce sebum-specific lipids. The mechanism by which the differentiation and lipid production are regulated in primary human sebocytes was next investigated.

### B.1 TGFβ Signaling is Active in Sebaceous Gland In Vivo and In Vitro

A study suggested TGFβ as a potential candidate for human sebocyte regulation [16]. TGFβ ligands bind to a bidimeric receptor complex composed of TGFβR1 and TGFβRII to phosphorylate and activate receptor-bound Smad (Smad2/3) transcription factors enabling them to translocate into the nucleus and regulate TGFβ-responsive genes [33]. TGFβRII is essential for the activation of the Smad2 pathway [20, 34]. Therefore the presence of TGFβRII and the functionality of the pathway in vivo and in vitro by the presence of phosphorylated Smad2/3 as readout for TGFβ activation was analyzed. Immunofluorescence has shown that TGFβRII is expressed throughout the sebaceous gland with the exception of the differentiated, lipid filled sebocytes present in the center of the gland (FIGS. 3a and 3a′). Further, it was determined that the TGFβ pathway is active in the gland in vivo by detecting the expression of nuclear phosphorylated Smad2 at the periphery and in the center of the gland but not in terminally differentiated sebocytes (FIGS. 3b and 3b′). In vitro, SSG3 sebocytes activate Smad2 when stimulated with exogenously recombinant TGFβ1 similarly to SEB-1 and NIKS (FIG. 3c).

### B.2 Effect of TGFβ Signaling on Sebocyte Differentiation Genes

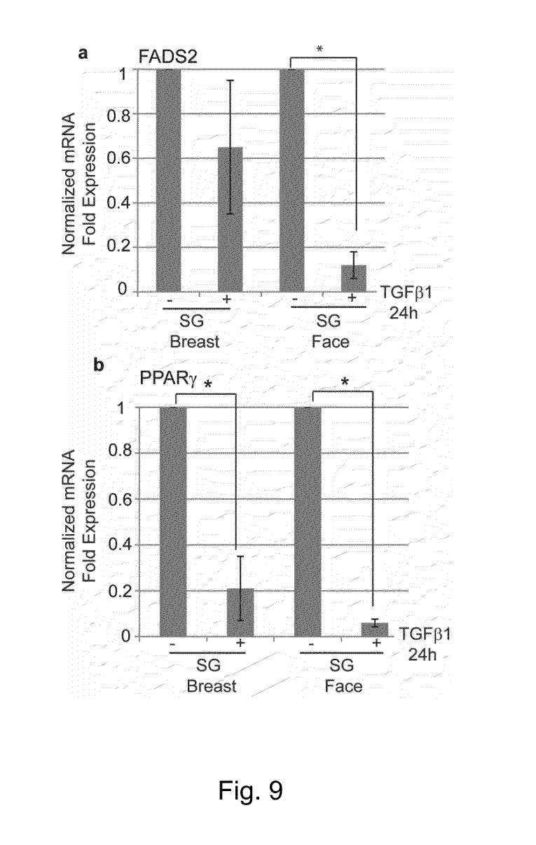

The effect of TGFβ signaling on sebocyte differentiation was next probed, by examining the expression of genes involved in lipogenesis upon treatment with TGFβ1. As shown in FIGS. 3a and b, when cells are stimulated with TGFβ1 for 24 h, the mRNA expression of FADS2 and PPARγ are significantly decreased in SSG3 cells compared to SEB-1 suggesting that TGFβ1 may prevent cell differentiation as well as in primary sebocytes derived from breast and face (FIG. 9). To test if those effects are TGFβRII-Smad2 dependent, shRNA was used to knockdown TGFβ receptor II, thus effectively inhibiting Smad2 phosphorylation [20].

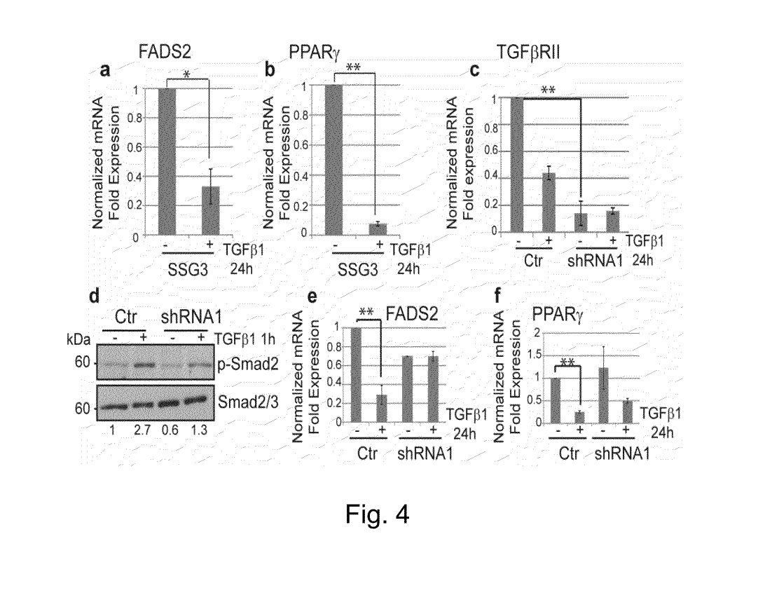

Similar results were obtained using two independent TGFβRII shRNA: TGFβRII expression was reduced in SSG3 cells (FIG. 4c). Phosphorylated-Smad2 was also decreased in shRNA expressing cells compared to controls after TGFβ activation (FIG. 4d) as expected. Also a decrease of TGFβRII was detected in control cells treated with TGFβ for 24 h (FIG. 4c) reflecting a possible degradation of the receptor [35]. It is shown that reduced TGFβRII expression inhibits the ability of SSG3 cells to decrease significantly FADS2 and PPARγ gene expression when cells are treated with TGFβ (FIGS. 4e and f).

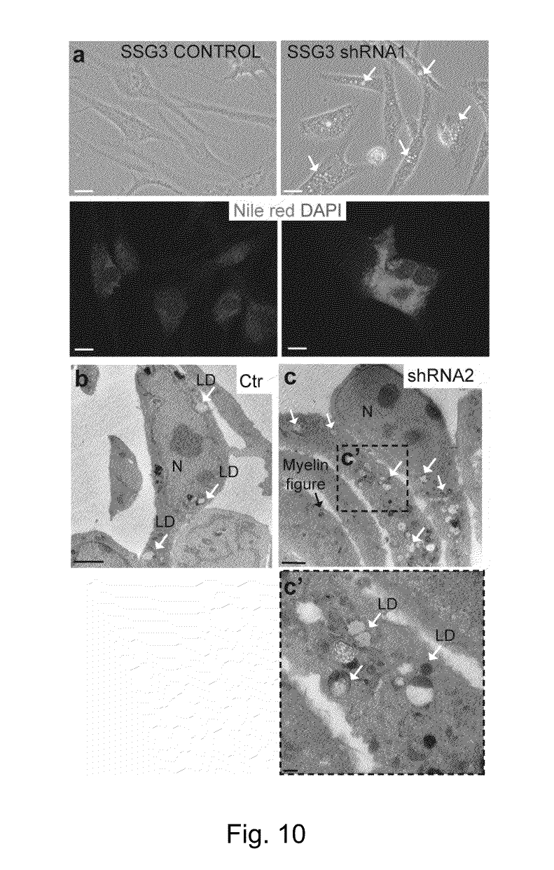

Determined next is how the inhibition of TGFβ signaling affects the functionality of SSG3 cells at a cellular level by analyzing the presence of cytoplasmic lipids in SSG3 shRNA expressing cells with reduced TGFβRII. TGFβRII depletion is associated with the increase of lipid inclusions positively stained with Nile red, Oil red O and identified by electron microscopy compare to SSG3 cells expressing a shRNA control (FIGS. 5b and c and FIG. 10). The lipids droplets labeled with Nile red have been also analyzed by flow cytometry (FIG. 5d). Similar to cells treated with linoleic acid, an increase in fluorescence and granularity (representing the lipid droplets) of the cells have been detected in SSG3 shRNA expressing cells with reduced TGFβRII compare to the shRNA control.

It has also been found that, whereas TGFβ1 treatment has no effect on the lipid production in the shRNA cells (FIG. 5b); it induces a decrease in lipid inclusion in SSG3 infected with a non-targeting shRNA control (FIG. 5a). These results suggest that inhibition of FADS2 and PPARγ at the transcriptional level is mediated via canonical Smad signal transduction. Together, the data place TGFβ signaling as an inhibitor of sebocyte differentiation that engages genes involved in the production of characteristic sebaceous lipids (FIG. 6).

### C. Discussion

Several lines of evidence suggested Transforming Growth Factor β (TGFβ) as a potential candidate for human sebocyte regulation [15, 16] but lack of primary human cultures has impaired an in-depth investigation of the molecular mechanism whereby TGFβ signaling controls sebaceous gland differentiation. The TGFβ pathway is ubiquitous and involved in the control of growth and differentiation of multiple cell and tissue types. TGFβ Receptor I (TGFβRI) and TGFβ Receptor II (TGFβRII), the two major receptors of the TGFβ signaling pathway, have been reportedly expressed in mouse sebaceous glands [17, 18]. In human and mouse epithelial cell lines, TGFβ acts as a potent inhibitor of proliferation mediated at least in part via down-regulation of cMyc expression [19, 20]. Intriguingly, c-Myc overexpression in mouse induced an increase of sebaceous gland size due to activation of sebocyte differentiation at the expense of hair differentiation [13, 21]. Moreover, disruption of epidermal Smad4, the common mediator of TGFβ signaling, leads to hyperplasia of inter-follicular epidermis, hair follicle, and sebaceous glands through c-Myc upregulation [22].

To determine the effect of TGFβ signaling on sebocyte differentiation, the effect of TGFβ ligands on the newly primary human sebocytes of the present disclosure was investigated. The findings show that activation of the TGFβ signaling pathway down-regulates the expression of genes involved in the production of characteristic sebaceous lipids. It was found that TGFβRII gene, which is essential for the activation of the Smad2 pathway, limits lipid production in primary human sebocytes. Together these findings show the role of TGFβ in the maintenance of human sebocytes in an undifferentiated state and highlight the relevance of this pathway in human sebaceous gland biology.

### D. Discussion of the Figures

FIG. 1. Fibronectin Mimics the Microenvironment and Allows Sebocytes to Grow In Vitro.

(a) Scalp sample (9 months old) before microdissection. (b) Isolated sebaceous gland. (c) Immunofluorescence staining on OCT sections of human scalp tissue showed that fibronectin (in red shown by white arrow in (c)) is expressed in the extracellular matrix surrounding the sebaceous gland. α6-integrin (in green, shown by white arrow in (c′)) marked the basal layer of the gland. Boxed area is magnified and shown to (c′). Scale bars, 20 μm (c, c′). (d) Schematic of new method to isolate and cultivate sebocytes. Scalp explants were placed between coverslips coated with fibronectin. Sebaceous gland cells SSG3 growing out of the explant (100× magnification). Abbreviations: SG, Sebaceous Gland, HF, Hair Follicle, FN, Fibronectin.

FIG. 2. Primary Sebocytes Isolated from Scalp Sebaceous Glands can Differentiate In Vitro and Produce Sebum-Characteristic Lipids.

(a) SSG3 expresses PPARγ but not Keratin 8 in contrast to SEB-1. (b-c) Real-time PCR shows that PPARγ is equally expressed in SEB-1 and SSG3 whereas FADS2 is more highly expressed in SSG3 cells than SEB-1. RNA from SEB-1 and SSG3 derived from the scalp explant at passage 3 were normalized to GAPDH expression. Data shown represent three independent experiments each performed in triplicate (mean+/−SD, n=3). *p-value<0.05 (unpaired two-tailed student's t test). (d) Cells were cultivated for 48 h with or without 0.1 mM of linoleic acid (LA). Differentiation through LA activation is followed by an increase in PPARγ expression in SSG3 cells. *p-value<0.05 paired two-tailed student's t test). (e) 24 h and 48 h of LA treatment induce a significant increase of FADS2 expression in SSG3 cells. *p-value<0.05 (paired two-tailed student's t test). (f) The Δ6 desaturase/FADS2 catalyzes the transformation of palmitic acid into sapienic acid. (g) Lipid analysis showing the percentage of Δ9 and Δ6 in the pellet of NIKS and SSG3 and the ratio Δ6/Δ9. (h) The sapienic acid (*) can be detected in SSG3 as in vivo sebum, whereas in NIKS, the palmitoleic acid (**) is the abundant lipid detected.

FIG. 3. TGFβ Signaling is Active in Sebaceous Gland In Vivo and In Vitro.

Sebaceous glands were sectioned in horizontal plane (red line in the diagram). (a) OCT sections of human scalp tissue stained with TGFβRII (red, shown by white arrow) show expression of the receptor throughout the sebaceous gland with the exception of the differentiated cells in the center. Boxed area is magnified and shown to (a′). (b) TGFβ pathway is active in vivo as denoted by the expression of nuclear phosphorylated Smad2 (red, shown by white arrow). α6: α6-integrin stains in green the basal layer of the sebaceous gland, shown by white arrow. Scale bars, 50 μm (a), 20 μm (a′, b, b′). Abbreviations: Epi, Epidermis; HF, Hair Follicle; SG, Sebaceous Gland. (c) The indicated sebocyte cultures were treated with 5 ng/ml of TGFβ1 ligand for one hour and whole cell extracts were examined by immunoblot to determine the activation of the TGFβ pathway.

FIG. 4. TGFβ Signaling Triggered Decreased Expression of Lipogenic Genes Through the TGFβRII-Smad2 Dependent Pathway.

(a, b) SSG3 cells were treated with 5 ng/ml of TGFβ1 for 24 hours and used for qPCR. Data were normalized to GAPDH expression and relative expression determined using untreated cells as a reference. FADS2 and PPARγ expression were found to be significantly downregulated in response to TGFβ1 treatment in SSG3 cells. (c) TGFβRII expression in SSG3 cells expressing TGFβRII shRNA1 and a control shRNA (Ctr) shows the efficiency of the knockdown. (d). Immunoblot confirms the decrease of p-Smad2 activity in shRNA expressing cells stimulated with TGFβ1 5 ng/ml for 1 h. Values, noted below the immunoblot, represent the relative density quantified with ImageJ using the ratio p-Smad2/Smad2/3 from each condition. (e, f). Decrease of FADS2 and PPARγ at the transcriptional level is mediated via canonical Smad signal transduction. The expression was normalized to control (Ctr) untreated. The significant decrease in PPARγ and FADS2 genes in control SSG3 cells after treatment with TGFβ1, is not detected in TGFβRII-deficient SSG3 cells. *p-value<0.05, **p-value<0.001 (paired two-tailed Student's t test).

FIG. 5. Inhibition of TGFβ Signaling Induces Lipogenesis in Primary SSG3 Cells.

(a, b) SSG3 cells stably expressing a shRNA against TGFβRII, show accumulation of lipid droplets on brightfield images (scale bars, 20 μm), by Nile red (scale bars, 20 μm) and Oil red O stainings (scale bars, 10 μm). White arrows show the presence of multiple lipid droplets in the shRNA expressing cells compared to the control (Ctr). 24 h of TGFβ1 (5 ng/ml) treatment decreases the basal level of lipid production in control cells but does not affect cells expressing the TGFβRII shRNA, mainly seen by Oil red O. (c) Electron microscopy showing the increase of lipid droplets in SSG3 cells (denoted by white arrows) expressing the shRNA compared to the control. Scale bars, 2 μm. LD, Lipid Droplets. N, Nucleus. (d) Flow cytometry of SSG3 cells expressing the shRNA labeled with Nile red. FL-1 measures the neutral lipids and SSC reflects the granularity of the cells. 10,000 cells have been acquired for each condition. As a positive control, SSG3 treated by 0.1 mM linoleic acid (LA) for 24 h show increase of fluorescence and granularity representing the lipid droplets. Note the increase of fluorescence and the increase of granularity in shRNA expressing TGFβRII compared to the cells expressing a shRNA control. Similar results were obtained similar with two different shRNA expressing TGFβRII (see FIG. 10).

FIG. 6. Model for the Role of TGFβ Signaling in Human Sebocyte Differentiation.

The sebaceous gland is composed of proliferative sebocytes at the exterior of the gland and differentiated sebocytes, filled with lipids in the center of the gland when they have reached their fully mature stage. The cellular environment surrounding the sebaceous gland is diverse including dermal fibroblasts, adipocytes that may be a source of TGFβ ligand to maintain sebocytes in an undifferentiated state by decreasing the expression of genes involved in lipid synthesis. APM: Arrector Pili Muscle, IRS: Inner Root Sheath, ORS: Outer Root Sheath.

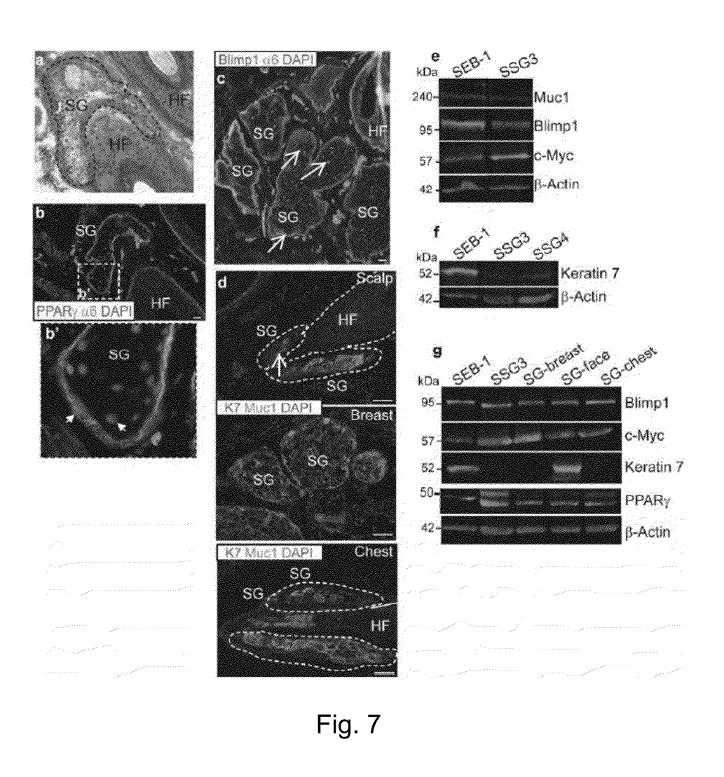

FIG. 7. Primary Human Sebocytes Derived from Scalp, Breast, Chest and Face Tissues Express Typical Sebocyte Markers.

(a) Hematoxylin and Eosin staining of the scalp sample. Scale bar, 50 μm. (b) Immunofluorescence staining showed that PPARγ (red, shown by white arrow in (b′)) is expressed in human sebaceous glands from the scalp explant at the periphery stained with a6-integrin (green, shown by white arrow) and at the center of the gland. Scale bar, 50 μm. Boxed area is magnified and shown to (b′). (c) Blimp1 (red, shown by white arrows) expression is mostly found in the differentiated cells of the sebaceous gland and in the inner root sheath of the hair follicle. α6-integrin (green, shown by white arrow) marked the basal layer of the gland. (d) Keratin 7 (red, shown by white arrow) expression varies depending on the location of the gland (scalp, breast and chest) as shown by immunofluorescence. (e-g) Sebocytes derived from the scalp, breast, chest and face explants expressed sebocytes markers by two-color immunoblot (Blimp1, c-Myc, Muc1, PPARγ and K7). SSG4 represents primary sebocytes derived from a four year old-scalp sample. Scale bars, 50 μm (b), 50 μm (c and d). Abbreviations: SG, Sebaceous Gland; HF, Hair Follicle; a6, a6-integrin; K7, Keratin 7.

FIG. 8. Primary Sebocytes can Differentiate In Vitro.

(a) Human scalp sections showing evidence of lipid accumulation (Nile red stain). Scale bar, 50 μm (b) SSG3 cells derived from the scalp explants were treated with 0.1 mM linoleic acid (LA) for 48 h to differentiate the cells and stained with Nile red to detect lipids. Images were taken with the same exposure time in untreated and linoleic acid-treated conditions. Brightfield pictures showed accumulation of cytoplasmic lipid droplets after linoleic acid treatment as denoted by the black arrows. Scale bars, 50 μm (c) Electron microscopy showing cytoplasmic lipid droplets in untreated sebocytes SSG3 derived from the scalp explants. Scale bar, 20 μm. Boxed area is magnified and shown to (c′) scale bar, 500 nm. (c″) After linoleic acid treatment increased high-electron density lipid droplets are detected in SSG3 cells and magnified in c′″. Scale bars for c″ and c′″ are 2 μm. Abbreviations: HF, Hair Follicle. SG, Sebaceous Gland. LD, Lipid Droplets. N, Nucleus. Mi, Mitochondria. RER, Rough Endoplasmic Reticulum. SER, Smooth Endoplasmic Reticulum.

FIG. 9. TGFβ Signaling Triggered Decreased Expression of Lipogenic Genes in Breast and Face-Derived Sebocytes.

RNA was isolated from sebocytes-derived from breast and face untreated or treated with 5 ng/ml of TGFβ1 for 24 h and used for real-time PCR. Two experiments were performed and all qPCR reactions were performed in triplicate. Data were normalized to GAPDH expression for each cell population and changes in relative expression were determined using untreated cells as a reference point. (a) FADS2 and (b) PPARγ expression was found to be decreased significantly in response to TGFβ1 treatment as shown in scalp-derived sebocytes (FIG. 4a-b) suggesting that the inhibitory effect of TGFβ is not due to the skin tissue type. *p-value<0.05 (paired two-tailed Student's t test).

FIG. 10. Inhibition of TGFβ Signaling Induces Lipogenesis in Primary SSG3 Cells.

(a) SSG3 cells, stably expressing a shRNA against TGFβRII (shRNA1), show accumulation of lipid droplets on brightfield image (white arrows) and by Nile red staining (shown in green) compared to cells infected with shRNA control. Scale bars, 20 μm. (b-c), Electron microscopy showing the increase of lipid droplets in SSG3 cells (denoted by white arrows) expressing the shRNA against TGFβRII (shRNA2) compared to the control. Myelin figures, which indicate lipids synthesis, are detected in SSG3 cells expressing the shRNA. Abbreviations: N, nucleus. LD, Lipid Droplets. Scale bars for b and c are 2 μm and 500 nm for c′.

### Example 3

### Screening of Compounds

The primary sebocytes will be used to test compounds known to be inhibitors or activators of lipogenesis, and identify test compounds that inhibit or activate lipogenesis, or change or alter the effects of an inhibitor or activator of lipogenesis.

Known inhibitors or activators of lipogenesis:

Androgen: sebum production is under androgen control, and an abnormal response of the pilosebaceous unit to androgens appears to be implicated in the pathogenesis of acne
5α-reductase inhibitor (use to treat androgenic alopecia): reduce lipogenesis
5α-DHT (di-hydrotestosterone) (androgen stimulates the activity of sebaceous gland in vivo): increase proliferation, increase lipogenesis.
DHEA (5-Dehydroepiandrosterone)(It is the major secretory steroidal product of the adrenal gland, acts on the androgen receptor, androgenic influence on sebaceous gland activity): increase lipogenesis
Cyproteron acetate (anti-androgen): decrease lipogenesis
Estrogens: Estradiol: decrease lipogenesis
Corticoids: Dexamethasone: decrease lipogenesis
Retinoids: Isotretinoin (13-cis retinoic acid): anti-proliferative effect on sebocytes, cell cycle arrest, apoptosis effect
PPAR Agonist: Rosglitazone: decrease lipogenesis

| Target | Reference name |
|---|---|
| Androgen metabolism | 5a- reductase inhibitor |
| Androgen | DHT |
| | DHEA |
| Anti androgen | Cyproteron acetate |
| Estrogens | Estradiol |
| Corticoids | Dexamethasone |
| Retinoids | Isotretinoin |
| PPAR Agonist | Rosglitazone |
| TGFβ pathway | TGF β1 ligand |

TGFβ1 ligand (10 ng/ml) can be tested, and should mimic the result using shRNA against TGFβRII, with an increase of lipid production after TGFβ inhibition.

### Materials and Methods:

The experiments are to be conducted on primary SSG3 cells, which were described in Examples 1 and 2. The experiments are to be conducted on primary SSG3 cells with and without induction with linoleic acid 0.1 mM for 48 h, as described in Example 2. Three different concentrations of each active compound are to be tested after two treatment times, 24 hrs and 48 hrs post linoleic acid treatment. The linoleic acid treatment is to be stopped when the active compound is added.

Following treatment, the effect on lipid production is to be assayed by two methods. In the first method, mRNA is to be extracted and real-time PCR is to be performed to analyze the expression of FADS2 and PPARγ shown to be increased after 48 h of linoleic acid treatment in SSG3. In the second method, the sebocytes are to be stained with Nile Red. The changes are to be quantified using FACS analysis. The corresponding fluorescence is to be measured at two different wavelengths (564 nm and 604 nm) which will allow the quantification of neutral lipid accumulation (representative of sebum lipids) and the presence of polar lipids (representative of phospholipids). The quantification is to be done by fluorescence activated cell sorting (FACS) using the default filter in a machine that has Yellow-Green laser to excite it.

As a control SSG3 is to be treated with TGFβ1 for 24 h, and a decrease in Nile red expression should be detected. The experiments will be done in triplicate to obtain significant data.

Comparison between two groups (untreated and treated) is to be performed using paired two-tailed student's t test. A p value<0.05 will be consider as significant.

Exemplary embodiments of the present disclosure include:

### Embodiment 1

A method of culturing primary sebocyte cells comprising culturing sebaceous glands sandwiched between pieces of glass in cell culture medium suitable for culturing sebocytes for a length of time sufficient for formation of sebocyte cells on said sebaceous glands.

### Embodiment 2

The method of Embodiment 1 wherein said pieces of glass are coated with an extracellular matrix protein.

### Embodiment 3

The method of Embodiments 1 or 2 wherein said extracellular matrix protein is fibronectin.

### Embodiment 4

The method of any one of Embodiments 1 to 3 wherein the sebaceous glands are from a human pediatric donor.

### Embodiment 5

The method of any one of Embodiments 1 to 4, further comprising, prior to culturing the sebaceous glands, obtaining a sample of skin, and removing sebaceous glands from the skin sample.

### Embodiment 6

The method of any one of Embodiments 1 to 5, wherein the sample of skin is from a human pediatric donor.

### Embodiment 7

The method of any one of Embodiments 1 to 6, wherein the cell culture medium comprises a basal medium, epidermal growth factor, cholera toxin, adenine, insulin, hydrocortisone, fetal bovine serum, and antibiotic/antimitotic.

### Embodiment 8

The method of any one of Embodiments 1 to 7, further comprising removing the sebocyte cells from the sebaceous gland, and culturing the sebocyte cells on glass coated with an extracellular matrix protein, in a medium suitable for culturing sebocytes.

### Embodiment 9

The method of any one of Embodiments 1 to 8, wherein the culture medium comprises a basal medium, epidermal growth factor, cholera toxin, adenine, insulin, hydrocortisone, fetal bovine serum, and antibiotic/antimitotic.

### Embodiment 10

The method of any one of Embodiments 1 to 9 wherein said extracellular matrix protein is fibronectin.

### Embodiment 11

A method of culturing primary sebocyte cells comprising culturing primary sebocyte cells on fibronectin coated glass in a medium comprising a basal medium, epidermal growth factor, cholera toxin, adenine, insulin, hydrocortisone, fetal bovine serum, and antibiotic/antimitotic.

### Embodiment 12

The method of Embodiment 11, wherein the primary sebocyte cells are derived from a human pediatric donor.

### Embodiment 13

An isolated population of cultured sebocyte cells obtained by the method of any one of claims 1-13.

### Embodiment 14

A method for identifying compounds that regulate lipogenesis comprising a) adding a test compound to the population of cultured sebocyte cells of Embodiment 13, and b) measuring the effect of the test compound on lipid production in the sebocyte cells.

### Embodiment 15

The method of Embodiment 14, wherein part of the population of cultured sebocytes is induced with linoleic acid prior to adding the test compound.

### Embodiment 16

The method of Embodiment 14 or 15, wherein measuring the effect of the test compound comprises measuring expression of FADS2 or PPARγ, or both.

The term “comprising” (and its grammatical variations) as used herein is used in the inclusive sense of “having” or “including” and not in the exclusive sense of “consisting only of” The terms “a”, “an” and “the” as used herein are understood to encompass the plural as well as the singular, unless indicated otherwise.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

Each of the claims defines a separate invention, which for infringement purposes is recognized as including equivalents to the various elements or limitations specified in the claims.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### REFERENCES

1. Smith K R, Thiboutot D M: “Thematic review series: skin lipids. Sebaceous gland lipids: friend or foe?” J Lipid Res 2008, 49(2):271-281.
2. Picardo M, Ottaviani M, Camera E, Mastrofrancesco A: “Sebaceous gland lipids”. Dermatoendocrinol 2009, 1(2):68-71.
3. Pochi P E, Strauss J S, Downing D T: “Age-related changes in sebaceous gland activity”. J Invest Dermatol 1979, 73(1):108-111.
4. Huang J T, Abrams M, Tlougan B, Rademaker A, Paller A S: “Treatment of Staphylococcus aureus colonization in atopic dermatitis decreases disease severity”. Pediatrics 2009, 123(5):e808-814.
5. Stenn K S: “Insights from the asebia mouse: a molecular sebaceous gland defect leading to cicatricial alopecia”. J Cutan Pathol 2001, 28(9):445-447.
6. Zheng Y, Eilertsen K J, Ge L, Zhang L, Sundberg J P, Prouty S M, Stenn K S, Parimoo S: “Scd1 is expressed in sebaceous glands and is disrupted in the asebia mouse”. Nat Genet 1999, 23(3):268-270.
7. Shizuru J A, Negrin R S, Weissman I L: “Hematopoietic stem and progenitor cells: clinical and preclinical regeneration of the hematolymphoid system.” Annu Rev Med 2005, 56:509-538.
8. Ronfard V, Rives J M, Neveux Y, Carsin H, Barrandon Y: “Long-term regeneration of human epidermis on third degree burns transplanted with autologous cultured epithelium grown on a fibrin matrix”. Transplantation 2000, 70(11):1588-1598.
9. Barrandon Y, Green H: “Three clonal types of keratinocyte with different capacities for multiplication”. Proc Natl Acad Sci USA 1987, 84(8):2302-2306.
10. Barrandon Y: “Crossing boundaries: stem cells, holoclones, and the fundamentals of squamous epithelial renewal”. Cornea 2007, 26(9 Suppl 1):S10-12.
11. Zouboulis C C, Seltmann H, Neitzel H, Orfanos C E: “Establishment and characterization of an immortalized human sebaceous gland cell line (S Z95)”. J Invest Dermatol 1999, 113(6): 1011-1020.
12. Thiboutot D, Jabara S, McAllister J M, Sivarajah A, Gilliland K, Cong Z, Clawson G: “Human skin is a steroidogenic tissue: steroidogenic enzymes and cofactors are expressed in epidermis, normal sebocytes, and an immortalized sebocyte cell line (SEB-1)”. J Invest Dermatol 2003, 120(6):905-914.
13. Lo Celso C, Berta M A, Braun K M, Frye M, Lyle S, Zouboulis C C, Watt F M: “Characterization of bipotential epidermal progenitors derived from human sebaceous gland: contrasting roles of c-Myc and beta-catenin”. Stem Cells 2008, 26(5):1241-1252.
14. Xia L, Zouboulis C C, Ju Q: “Culture of human sebocytes in vitro”. Dermatoendocrinol 2009, 1(2):92-95.
15. Fume M, Kato M, Nakamura K, Nashiro K, Kikuchi K, Okochi H, Miyazono K, Tamaki K: “Dysregulated expression of transforming growth factor beta and its type-I and type-II receptors in basal-cell carcinoma”. Int J Cancer 1997, 71(4):505-509.
16. Downie M M, Sanders D A, Kealey T: “Modelling the remission of individual acne lesions in vitro”. Br J Dermatol 2002, 147(5):869-878.
17. Wollina U, Lange D, Funa K, Paus R: “Expression of transforming growth factor beta isoforms and their receptors during hair growth phases in mice”. Histol Histopathol 1996, 11(2):431-436.
18. Paus R, Foitzik K, Welker P, Bulfone-Paus S, Eichmuller S: “Transforming growth factor-beta receptor type I and type II expression during murine hair follicle development and cycling”. J Invest Dermatol 1997, 109(4):518-526.
19. Pietenpol J A, Stein R W, Moran E, Yaciuk P, Schlegel R, Lyons R M, Pittelkow M R, Munger K, Howley P M, Moses H L: “TGF-beta 1 inhibition of c-myc transcription and growth in keratinocytes is abrogated by viral transforming proteins with pRB binding domains”. Cell 1990, 61(5):777-785.
20. Guasch G, Schober M, Pasolli H A, Conn E B, Polak L, Fuchs E: “Loss of TGFbeta signaling destabilizes homeostasis and promotes squamous cell carcinomas in stratified epithelia”. Cancer Cell 2007, 12(4):313-327.
21. Arnold I, Watt F M: “c-Myc activation in transgenic mouse epidermis results in mobilization of stem cells and differentiation of their progeny”. Curr Biol 2001, 11(8):558-568.
22. Yang L, Wang L, Yang X: “Disruption of Smad4 in mouse epidermis leads to depletion of follicle stem cells”. Mol Biol Cell 2009, 20(3):882-890.
23. Rosenfield R L, Deplewski D, Kentsis A, Ciletti N: “Mechanisms of androgen induction of sebocyte differentiation”. Dermatology 1998, 196(1):43-46.
24. Allen-Hoffmann B L, Schlosser S J, Ivarie C A, Sattler C A, Meisner L F,
O'Connor S L: “Normal growth and differentiation in a spontaneously immortalized near-diploid human keratinocyte cell line”, NIKS. J Invest Dermatol 2000, 114(3):444-455.
25. Troyanovsky S M, Guelstein V I, Tchipysheva T A, Krutovskikh V A, Bannikov G A: “Patterns of expression of keratin 17 in human epithelia: dependency on cell position”. J Cell Sci 1989, 93 (Pt 3):419-426.
26. Magnusdottir E, Kalachikov S, Mizukoshi K, Savitsky D, Ishida-Yamamoto A, Panteleyev A A, Calame K: “Epidermal terminal differentiation depends on B lymphocyte-induced maturation protein-1”. Proc Natl Acad Sci USA 2007, 104(38):14988-14993.
27. Braun K M, Niemann C, Jensen U B, Sundberg J P, Silva-Vargas V, Watt F M: “Manipulation of stem cell proliferation and lineage commitment: visualisation of label-retaining cells in wholemounts of mouse epidermis”. Development 2003, 130(21):5241-5255.
28. Rosenfield R L, Kentsis A, Deplewski D, Ciletti N: “Rat preputial sebocyte differentiation involves peroxisome proliferator-activated receptors”. J Invest Dermatol 1999, 112(2):226-232.
29. Ge L, Gordon J S, Hsuan C, Stenn K, Prouty S M: “Identification of the delta-6 desaturase of human sebaceous glands: expression and enzyme activity”. J Invest Dermatol 2003, 120(5):707-714.
30. Zouboulis C C, Angres S, Seltmann H: “Regulation of stearoyl-coenzyme A desaturase and fatty acid delta-6 desaturase-2 expression by linoleic acid and arachidonic acid in human sebocytes leads to enhancement of proinflammatory activity but does not affect lipogenesis”. Br J Dermatol 2011, 165(2):269-276.
31. Pappas A, Anthonavage M, Gordon J S: “Metabolic fate and selective utilization of major fatty acids in human sebaceous gland”. J Invest Dermatol 2002, 118(1):164-171.
32. Ottaviani M, Camera E, Picardo M: “Lipid mediators in acne”. Mediators Inflamm 2010, 2010.
33. Massague J, Gomis R R: “The logic of TGF-beta signaling”. FEBS Lett 2006, 580(12):2811-2820.
34. Wrana J L, Attisano L, Wieser R, Ventura F, Massague J: “Mechanism of activation of the TGF-beta receptor”. Nature 1994, 370(6488):341-347.
35. Mitchell H, Choudhury A, Pagano R E, Leof E B: “Ligand-dependent and -independent transforming growth factor-beta receptor recycling regulated by clathrin-mediated endocytosis and Rab11”. Mol Biol Cell 2004, 15(9):4166-4178.
36. Eisinger M, Li W H, Rossetti D D, Anthonavage M, Seiberg M: “Sebaceous gland regeneration in human skin xenografts”. J Invest Dermatol 2010, 130(8):2131-2133.
37. Rendl M, Lewis L, Fuchs E: “Molecular dissection of mesenchymal-epithelial interactions in the hair follicle”. PLoS Biol 2005, 3(11):e331.
38. Wong T, McGrath J A, Naysaria H: “The role of fibroblasts in tissue engineering and regeneration”. Br J Dermatol 2007, 156(6):1149-1155.
39. Nolte S V, Xu W, Rennekampff H O, Rodemann H P: “Diversity of fibroblasts—a review on implications for skin tissue engineering”. Cells Tissues Organs 2008, 187(3):165-176.
40. Fujiwara H, Ferreira M, Donati G, Marciano D K, Linton J M, Sato Y, Hartner A, Sekiguchi K, Reichardt L F, Watt F M: “The basement membrane of hair follicle stem cells is a muscle cell niche”. Cell 2011, 144(4):577-589.
41. Poblet E, Jimenez F, Ortega F: “The contribution of the arrector pili muscle and sebaceous glands to the follicular unit structure”. J Am Acad Dermatol 2004, 51(2):217-222.
42. Chronnell C M, Ghali L R, Ali R S, Quinn A G, Holland D B, Bull J J, Cunliffe W J, McKay I A, Philpott M P, Muller-Rover S: “Human beta defensin-1 and -2 expression in human pilosebaceous units: upregulation in acne vulgaris lesions”. J Invest Dermatol 2001, 117(5):1120-1125.
43. Nagy I, Pivarcsi A, Kis K, Koreck A, Bodai L, McDowell A, Seltmann H, Patrick S, Zouboulis C C, Kemeny L: “Propionibacterium acnes and lipopolysaccharide induce the expression of antimicrobial peptides and proinflammatory cytokines/chemokines in now abandoned human sebocytes”. Microbes Infect 2006, 8(8):2195-2205.
44. Lee D Y, Yamasaki K, Rudsil J, Zouboulis C C, Park G T, Yang J M, Gallo R L: “Sebocytes express functional cathelicidin antimicrobial peptides and can act to kill propionibacterium acnes”. J Invest Dermatol 2008, 128(7):1863-1866.
45. Zouboulis C C: “Propionibacterium acnes and sebaceous lipogenesis: a love-hate relationship?”, J Invest Dermatol 2009, 129(9):2093-2096.
46. Kawsar H I, Ghosh S K, Hirsch S A, Koon H B, Weinberg A, Jin G: “Expression of human beta-defensin-2 in intratumoral vascular endothelium and in endothelial cells induced by transforming growth factor beta”. Peptides 2010, 31(2): 195-201.
47. Ishinaga H, Jono H, Lim J H, Kweon S M, Xu H, Ha U H, Koga T, Yan C, Feng X H, Chen L F et al: “TGF-beta induces p65 acetylation to enhance bacteria-induced N F-kappaB activation”. EMBO J 2007, 26(4):1150-1162.
48. Runck L A, Kramer M, Ciraolo G, Lewis A G, Guasch G: “Identification of epithelial label-retaining cells at the transition between the anal canal and the rectum in mice”. Cell Cycle 2010, 9(15):3039-3045.
49. Stoffel W, Holz B, Jenke B, Binczek E, Gunter R H, Kiss C, Karakesisoglou I, Thevis M, Weber A A, Arnhold S, Addicks K. “Delta6-desaturase (FADS2) deficiency unveils the role of omega3- and omega6-polyunsaturated fatty acids”. EMBO J 2008; 27:2281-92.

1. A method of culturing primary sebocyte cells comprising culturing sebaceous glands sandwiched between pieces of glass in cell culture medium suitable for culturing sebocytes for a length of time sufficient for formation of sebocyte cells on said sebaceous glands.

2. The method of claim 1 wherein said pieces of glass are coated with an extracellular matrix protein.

3. The method of claim 2 wherein said extracellular matrix protein is fibronectin.

4. The method of claim 1 wherein the sebaceous glands are from a human pediatric donor.

5. The method of claim 1, further comprising, prior to culturing the sebaceous glands, obtaining a sample of skin, and removing sebaceous glands from the skin sample.

6. The method of claim 5, wherein the sample of skin is from a human pediatric donor.

7. The method of claim 1, wherein the cell culture medium comprises a basal medium, epidermal growth factor, cholera toxin, adenine, insulin, hydrocortisone, fetal bovine serum, and antibiotic/antimitotic.

8. The method of claim 1, further comprising removing the sebocyte cells from the sebaceous gland, and culturing the sebocyte cells on glass coated with an extracellular matrix protein, in a medium suitable for culturing sebocytes.

9. The method of claim 8, wherein the culture medium comprises a basal medium, epidermal growth factor, cholera toxin, adenine, insulin, hydrocortisone, fetal bovine serum, and antibiotic/antimitotic.

10. The method of claim 8 wherein said extracellular matrix protein is fibronectin.

11. A method of culturing primary sebocyte cells comprising culturing primary sebocyte cells on fibronectin coated glass in a medium comprising a basal medium, epidermal growth factor, cholera toxin, adenine, insulin, hydrocortisone, fetal bovine serum, and antibiotic/antimitotic.

12. The method of claim 11, wherein the primary sebocyte cells are derived from a human pediatric donor.

13. An isolated population of cultured sebocyte cells obtained by the method of claim 1.

14. As isolated population of cultured sebocyte cells obtained by the method of claim 11.

15. A method for identifying compounds that regulate lipogenesis comprising a) adding a test compound to the population of cultured sebocyte cells of claim 13, and b) measuring the effect of the test compound on lipid production in the sebocyte cells.

16. The method of claim 15 wherein part of the population of cultured sebocytes is induced with linoleic acid prior to adding the test compound.

17. The method of claim 15 wherein measuring the effect of the test compound comprises measuring expression of FADS2 or PPARγ, or both.

18. A method for identifying compounds that regulate lipogenesis comprising a) adding a test compound to the population of cultured sebocyte cells of claim 14, and b) measuring the effect of the test compound on lipid production in the sebocyte cells.

19. The method of claim 18 wherein part of the population of cultured sebocytes is induced with linoleic acid prior to adding the test compound.

20. The method of claim 18 wherein measuring the effect of the test compound comprises measuring expression of FADS2 or PPARγ, or both.
